(19) United States
(12) Patent Application Publication
(10) Pub. No.: US 2025/0144384 A1
(43) Pub. Date: May 8, 2025

(54) INGESTIBLE DEVICE WITH DETACHMENT OF TISSUE PENETRATING MEMBER

(71) Applicant: Novo Nordisk A/S, Bagsvaerd (DK)

(72) Inventors: Aghiad Ghazal, Koebenhavn SV (DK); Brian Jensen, Broenshoej (DK); Maria Pereverzina, Koebenhavn SV (DK); Drago Sticker, Malmoe (SE); Jacob Pyung Hwa Jepsen, Frederiksberg (DK); Brian Mouridsen, Fredensborg (DK); Cody Edward Cleveland, Koebenhavn K (DK); Mette Poulsen, Glostrup (DK); Anders Hagedal Uhrenfeldt, Alleroed (DK); Anders Hammelev Marstrand, Koebenhavn N (DK); Noekkvi Steinn Sigurdarson, Koebenhavn N (DK); Adam Bo Mandel, Keobenhavn N (DK); Camilla Sandfeld, Koebenhavn NV (DK); Jesper Peter Windum, Hilleroed (DK); Nicolai Aaberg Hansen, Oelstykke (DK); Kristina Ray Rivera

(21) Appl. No.: 18/832,669

(22) PCT Filed: Jan. 31, 2024
(86) PCT No.: PCT/EP2023/052217

§ 371 Date: Jul. 24, 2024

(30) Foreign Application Priority Data
Jan. 31, 2022 (EP) 22154390.3
Aug. 17, 2022 (EP) 22190669.6
Aug. 17, 2022 (EP) 22190670.4
Dec. 1, 2022 (EP) 22210928.2
Dec. 1, 2022 (EP) 22210929.0

(51) Int. Cl. A61M 31/00

(52) U.S. Cl. CPC A61M 31/002; A61M 2202/0007; A61M 2202/04; A61M 2202/064; A61M 2205/02; A61M 2210/1042

(57) ABSTRACT

An ingestible device (200), comprising: a housing (210, 220) comprising a stop geometry (228) at an exit hole (224); a tissue penetrating member (230) and an actuator that comprises: a pushing portion (260) configured for axial movement from a first to a second position, the pushing portion configured to move the tissue penetrating member from within the housing to a lodged position in tissue, and a holder portion (270) coupled with the pushing portion (260) for initial axial movement with the pushing portion. In the first position the holder portion releasably holds the tissue penetrating member (230) in axial retaining engagement. As the pushing portion (260) moves towards the second position, the tissue penetrating member (230) moves through the exit hole (224) until the holder portion (270) enters into engagement with the stop geometry (228). The pushing portion (260) moves further to release and advance the tissue penetrating member (230).

The present invention relates to ingestible devices adapted for being swallowed into a lumen of a patient and having a tissue penetrating member being shaped to penetrate tissue of a lumen wall.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise, since protein-based drugs will be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to oral dosing of active agents and addressing one or more of the above challenges include WO 2018/213600 A1 and WO 2017/156347 A1.

Ingestible capsules have been proposed comprising a delivery member formed as a solid formed from a preparation comprising a therapeutic payload, wherein the delivery member is forced from the capsule and into tissue of the lumen wall for delivering the payload. The payload is inserted into tissue and will over time dissolve and be absorbed into the body of the patient. Even though the capsule may be able to properly orient relative to a target site it can still move to another location after deployment of the payload. This introduces the risk that the payload will be partly or fully removed from the target site due to movement of the capsule.

Having regard to the above, it is an object of the present invention to provide an ingestible device for swallowing into a lumen of a gastrointestinal tract, and which to a high degree effectively and reliably ensures proper deposition of the delivery member into tissue.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention an ingestible device is provided suitable for swallowing into a lumen of a gastrointestinal tract of a patient, the lumen having a lumen wall, the ingestible device comprising:
a housing defining an internal hollow and comprising a stop geometry arranged within the internal hollow and an exit hole,
a tissue penetrating member disposable in the housing, the tissue penetrating member having a tissue penetrating first end, a second end opposite the first end and a radially outwards facing surface between the first end and the second end, and
an actuator arrangement comprising:
a) a pushing portion configured for movement along an axis from a first position and into a second position, the pushing portion being configured for providing a force onto the tissue penetrating member to move the tissue penetrating member from an initial position within the housing to a lodged position where at least a portion of the tissue penetration member is external to the housing and at least partially lodged in tissue of the lumen wall, and
b) a holder portion coupled with the pushing portion for initial axial movement with the pushing portion, wherein when the pushing portion assumes the first position the holder portion releasably holds the tissue penetrating member in axial retaining engagement,
wherein, as the pushing portion moves towards the second position, the tissue penetrating member is moved into tissue until the holder portion enters into engagement with the stop geometry of the housing thereby axially stopping the holder portion, whereafter the pushing portion moves further to release the axial retaining engagement and advance the tissue penetrating member into its lodged position.

In accordance with the first aspect, the tissue penetrating member is effectively detached from the pushing portion at the point in time relative the moving of tissue penetrating member from the initial position within the housing to the lodged position. Hence, the risk associated with the housing of the ingestible device being accidentally moved relative to the target tissue, which would potentially move the tissue penetrating member away from the designated position, becomes less critical.

Also, for applications wherein the tissue penetrating member forms part of, or comprises, a therapeutic payload, compared to prior art solutions wherein only a portion of a tissue penetrating member is lodged within tissue, the solution according to the first aspect enables a larger percentage of the therapeutic payload to be available for being lodged in tissue and for subsequent release into the blood stream.

In some embodiments, the stop geometry is formed by a housing shell. In other embodiments, the stop geometry is formed by a component which is fixedly mounted relative to a housing shell. In different embodiments, the holder portion may be configured to enter into direct engagement with the stop geometry of the housing. In other embodiments, the holder portion enters into engagement with the stop geometry of the housing via one or more intermediate components.

In some embodiments, when the tissue penetrating member assumes the initial position, the holder portion assumes a start position, and wherein the holder portion is moved from the start position towards the stop geometry by being displaced by slaved movement relative to the pushing portion.

In some forms the holder portion, when assuming the start position, is in friction engagement with the pushing portion, and wherein pushing portion overcomes the friction engagement when the holder portion enters into engagement with the stop geometry.

In further forms the holder portion, when assuming the start position, releasably engages the pushing portion, such as by a friction engagement or a snap engagement, and wherein the pushing portion releases from engagement with the holder portion when the holder portion enters into engagement with the stop geometry.

In further forms of the ingestible device the holder portion is formed as a sleeve that, when the holder portion is slaved relative to the pushing portion, interconnects the pushing portion with the tissue penetrating member.

The holder portion may in some embodiments comprise at least one radially resilient gripping member providing a radially inwards directed force onto the tissue penetrating member, wherein the radially resilient gripping member cooperates with the stop geometry of the housing to release the radially inwards directed force upon the holder portion engaging the stop geometry.

In some further embodiments the holder portion defines a distal facing end surface and wherein, when the holder portion holds the tissue penetrating member in axial retaining engagement, the distal facing end surface at least partially encircles the tissue penetration member while the tissue penetrating member extends distally from the distal facing end surface of the holder portion.

In still further forms the holder portion comprises at least one piercing portion that protrudes axially from the distal facing end surface of the holder portion, the at least one piercing portion extending axially past the first end of the tissue penetrating member. In different embodiments, when the pushing portion assumes the first position, the piercing portion protrudes axially distally past the distal end of the tissue penetrating member, such as by protruding a distance in the range from 0.5 mm to 3 mm further distally than the first end of the tissue penetrating member.

In some embodiments, the pushing portion and the holder portion are formed as a unitarily formed member formed from a deformable material and wherein the holder portion deforms when engaging the stop geometry to release the retention force.

In still further embodiments, a further stop feature is associated with the housing, said further stop feature being configured to block axial movement of the pushing portion when the pushing portion assumes the second position. In some embodiments, the further stop feature is formed by a housing shell. In other embodiments, the further stop feature is formed by a component which is fixedly mounted relative to a housing shell. In different embodiments, the pushing portion may be configured to enter into direct engagement with the further stop feature of the housing. In other embodiments, the pushing portion enters into engagement with the further stop feature of the housing via one or more intermediate components.

The housing may be formed to comprise an exterior surface portion surrounding the exit hole, wherein the exit hole permits the tissue penetrating member, the holder portion and the pushing portion to protrude through the exit hole, and wherein the pushing portion in its second position pushes the first end of the tissue penetrating member a predefined distance from the exterior surface portion, said pre-defined distance selected between 3 and 7 mm, such as between 4 and 6 mm and such as between 4.5 and 5.5 mm.

The housing may be so configured that an exterior surface portion surrounds the exit hole, the exit hole permits the tissue penetrating member and the pushing portion to protrude through the exit hole, and wherein the pushing portion in its second position pushes the second end of tissue penetrating member a predefined distance from the exterior surface portion, said predefined distance selected between 1 and 5 mm, such as between 2 and 4.5 mm and such as between 2.5 and 4 mm.

In some further forms, when the tissue penetrating member assumes the initial position, the tissue penetrating first end is axially separated relative to an exterior surface portion surrounding the exit hole by a separating distance, thereby enabling the tissue penetrating member to be advanced towards tissue at a target location by an acceleration stroke corresponding to a separating distance selected in the range from 0.5 mm to 3 mm, such as in the range from 1 mm to 2.5 mm.

In further embodiments, the pushing portion and the holder portion comprises a protruding section configured to protrude through the exit hole, and wherein at least a portion of the said protruding sections are made from a material configured to change shape, such as by degrading, softening, or swelling, when exposed to gastric fluid.

In some forms the tissue penetrating member is a solid formed partly or entirely from a preparation comprising a therapeutic payload, wherein the tissue penetrating member is made from a dissolvable material that dissolves when inserted into tissue of the lumen wall to deliver at least a portion of the therapeutic payload into tissue.

In alternative forms, an exterior portion of the tissue penetrating member defines an enclosure, and wherein a preparation comprising a therapeutic active substance forms a liquid, gel or powder accommodated within the enclosure.

In some embodiments the actuator arrangement comprises an energy source configured for powering the tissue penetrating member for being advanced from the housing and into the lodged position in the lumen wall and wherein a trigger arrangement is coupled to the actuator arrangement for initiating release of energy from the energy source thereby driving the pushing portion from the first position to the second position.

In some embodiments the actuator arrangement comprises a drive spring, such as a compression spring or a tension spring, the spring being strained or configured for being strained for powering the pushing portion for movement from the first position to the second position.

In some forms the ingestible device is configured as a self-orienting capsule device, wherein when the self-orienting capsule device is at least partially supported by the tissue of the lumen wall, the self-orienting capsule device orients in a direction to allow the tissue penetrating member to be inserted into the lumen wall.

In some further forms, when the tissue penetrating member assumes the initial position, the tissue penetrating first end is axially separated from the exit hole by a separating distance, thereby enabling the tissue penetrating member to be advanced towards the exit hole by an acceleration stroke corresponding to the separating distance. Exemplary separating distances may be provided as larger than 0.5 mmm, such as larger than 1 mm, such as larger than 1.5 mm, such as larger than 2 mm, such as larger than 3 mm.

In some forms, the pushing portion and/or the tissue penetrating member is configured for movement along an axis. In other forms, the said components may be moved along a non-linear path, such as a curved path.

In some forms, the actuator arrangement comprises a hub that comprises at least one pair of a latch and a retainer portion structured to maintain the hub in a pre-actuation configuration. For each pair of latch and retainer portion the ingestible device defines a dissolvable firing member, the dissolvable firing member being at least partially dissolved in a fluid, such as a biological fluid, a retainer portion comprised by one of the housing and the hub, and a deflectable latch comprised by the other of the housing and the hub. The deflectable latch may be configured for lateral movement relative to the axis, and the deflectable latch defines a first surface with a blocking portion, and a support surface disposed oppositely to the first surface and configured for interacting with the dissolvable firing member. In the pre-actuation configuration, the blocking portion of the deflectable latch engages the retainer portion in a latching engagement, and the support surface of the deflectable latch interacts with the dissolvable firing member to restrict movement of the deflectable latch thereby preventing release of the latching engagement. In an actuated configuration wherein the dissolvable firing member has become at least partially dissolved, the deflectable latch is allowed to move thereby releasing the latching engagement between the blocking portion of the deflectable latch and the retainer portion to allow the energy source to actuate/fire the hub.

By this arrangement, instead of having a dissolvable member that carries the whole power or load of the energy source, the dissolvable part is designed to simply block a mechanical activation system. The mechanical activation system may be designed to rely on parts made from a suitable high-strength material, such as plastic, and do not leave any undissolved pieces that potentially could jam the mechanical activation system.

In exemplary embodiments, the deflectable latch is configured for radial movement relative to the axis. In some examples the firing axis and the hub movement is linear. In other exemplary embodiments, the firing axis may be not linear, e.g. the firing trajectory of the hub may be arcuate or curved, or may include arcuate or curved trajectories. In accordance herewith, the latch may be configured for lateral movement relative to the trajectory of the hub to release the hub.

In exemplary embodiments a plurality of pairs of latch and retainer portions, such as two, three, four, five or more pairs of latch and retainer portions are provided, the pairs of latch and retainer portions being disposed equally around the axis.

In some embodiments said dissolvable firing member is common to all pairs of latch and retainer portions.

In further embodiments, the dissolvable firing member is arranged along the axis, wherein the at least one pair of latch and retainer portion is disposed radially outside of the dissolvable firing member.

In other variants one or more dissolvable firing members is/are disposed, such as in a ring-shaped configuration around the axis, wherein the one or more dissolvable firing members encircle the at least one pair of latch and retainer portion.

The capsule may comprise one or more openings to allow a biologic fluid, such as gastric fluid, to enter the capsule for dissolving the dissolvable firing member(s).

In some embodiments, the energy source is or comprises at least one spring configured as a drive spring. Exemplary springs include a compression spring, a torsion spring, a leaf spring or a constant-force spring. The spring may either be strained or configured for being strained for powering the hub. Other non-limiting exemplary types of energy sources for the actuator include compressed gas actuators or gas generators. In some embodiments, in the pre-actuation configuration, the energy source exerts a load onto the hub thereby biasing the hub along the axis. In other embodiments the energy source is configured to exert a load onto the hub only upon triggering of a trigger member or mechanism of the ingestible device.

In exemplary embodiments, the ingestible device is configured for swallowing by a patient and travelling into a lumen of a gastrointestinal tract of a patient, such as the stomach, the small intestines or the large intestines. The capsule of the device may be shaped and sized to allow it to be swallowed by a subject, such as a human.

In still further exemplary embodiments, the ingestible device is configured as a self-righting capsule, wherein when the self-righting capsule is at least partially supported by the tissue of the lumen wall, the self-righting capsule orients in a direction to allow the delivery member to be inserted into the lumen wall to deliver at least a portion of the therapeutic payload into the tissue. The ingestible device may in certain embodiments be configured as a self-righting capsule device having a geometric center and a center of mass offset from the geometric center along the axis, wherein when the capsule device is supported by the tissue of the lumen wall while being oriented so that the centre of mass is offset laterally from the geometric center the capsule device experiences an externally applied torque due to gravity acting to orient the capsule device with the axis oriented along the direction of gravity to enable the delivery member to interact with the lumen wall at the target location.

In certain embodiments, the self-righting capsule may be configured to define a monostatic body, such as a Gömböc or Gömböc-type shape that, when placed on a surface in any orientation other than a single stable orientation of the body, the body will tend to reorient to its single stable orientation. In typical embodiments, the single stable orientation of the body aligns the exit hole so that the exit hole faces vertically downward towards supporting tissue at a target location.

In some embodiments, the actuation of the actuator arrangement is controlled by a gastrointestinal tract environmentally-sensitive mechanism.

The GI tract environmentally-sensitive mechanism may comprise a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

In alternative forms, the trigger arrangement may also be or include an electronic trigger.

In a second aspect of the invention an ingestible device is provided suitable for swallowing into a lumen of a gastrointestinal tract of a patient, the lumen having a lumen wall, the ingestible device comprising:
a housing defining an internal hollow and comprising a stop geometry arranged within the internal hollow,
a tissue penetrating member disposable in the housing, the tissue penetrating member having a tissue penetrating first end, a second end opposite the first end and a radially outwards facing surface between the first end and the second end, and
an actuator arrangement comprising:
a) a pushing portion configured for movement from a first position and into a second position, the pushing portion being configured for providing a force onto the tissue penetrating member to move the tissue penetrating member from an initial position within the housing to a lodged position where at least a portion of the tissue penetration member is external to the housing and at least partially lodged in tissue of the lumen wall, and
b) a holder portion which, when the pushing portion assumes the first position, releasably holds the tissue penetrating member by exerting a retention force, such as on the radially outwards facing surface of the tissue penetrating member,
wherein, as the pushing portion moves towards the second position, the holder portion enters into engagement with the stop geometry of the housing to initiate release of the retention force, whereafter the pushing portion moves further towards the second position until the tissue penetration member assumes its lodged position.

Any of the features defined in connection with the first aspect may in alternative embodiments be combined with the second embodiment.

By the above arrangements an orally administered drug substance can be delivered safely and reliably into the stomach wall or intestinal wall of a living mammal subject. The drug substance may e.g. be in the form of a solid, an encapsulated solid, a liquid, a gel or a powder, or any combination thereof.

As used herein, the terms “drug”, “drug substance” or “payload” is meant to encompass any drug formulation capable of being delivered into or onto the specified target site. The drug may be a single drug compound or a premixed or co-formulated multiple drug compound. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIGS. 1a and 1b show cross-sectional views of a prior art capsule device 100, the device assuming a pre-actuation configuration and an actuated configuration, respectively,

FIGS. 2a and 2b each shows a cross-sectional front view of a first embodiment capsule device 200 in accordance with the invention, the device assuming a pre-actuation configuration and an actuated configuration, respectively,

FIG. 2c shows a detailed view of a subassembly of the first embodiment capsule device 200 providing a push rod 260, a holder sleeve 270 and a payload portion 230,

FIGS. 3a and 3b each shows a cross-sectional front view of a second embodiment capsule device 300 in accordance with the invention, the device assuming a pre-actuation configuration and an actuated configuration, respectively,

FIG. 3c shows a detailed view of select components of second embodiment capsule device 300 in a state immediately prior to the actuated configuration

FIGS. 4a and 4b are cross-sectional front views of a third embodiment capsule device 400 in accordance with the invention, the device assuming a pre-actuation configuration and an actuated configuration, respectively,

FIG. 4c is a perspective view of an upper capsule part 410 of the capsule device 400 of FIGS. 4a and 4b.

FIG. 4d is a perspective view of an actuator hub upper part 451 of the capsule device 400 of FIGS. 4a and 4b,

FIGS. 5a and 5b show detailed perspective views of a subassembly of a fourth embodiment capsule device, the subassembly consisting of a push rod 560, a holder sleeve 570 and a payload portion 530, with FIG. 5a showing the pre-actuation configuration and FIG. 5b showing the actuated configuration, respectively,

FIG. 5c are three cross-sectional and side views of the fourth embodiment subassembly in the pre-actuation configuration corresponding to FIG. 5a,

FIG. 5d are three cross-sectional and side views of the fourth embodiment subassembly in the actuated configuration corresponding to FIG. 5b,

FIGS. 6a and 6b show detailed perspective views of a subassembly of a fifth embodiment capsule device, the subassembly consisting of a push rod 660, a holder sleeve 670 and a payload portion 630, with FIG. 6a showing the pre-actuation configuration and FIG. 6b showing the actuated configuration, respectively,

FIG. 6c are two side views of the fifth embodiment subassembly in the pre-actuation configuration corresponding to FIG. 6a, and

FIG. 6d are two side views of the fifth embodiment subassembly in the actuated configuration corresponding to FIG. 6b.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

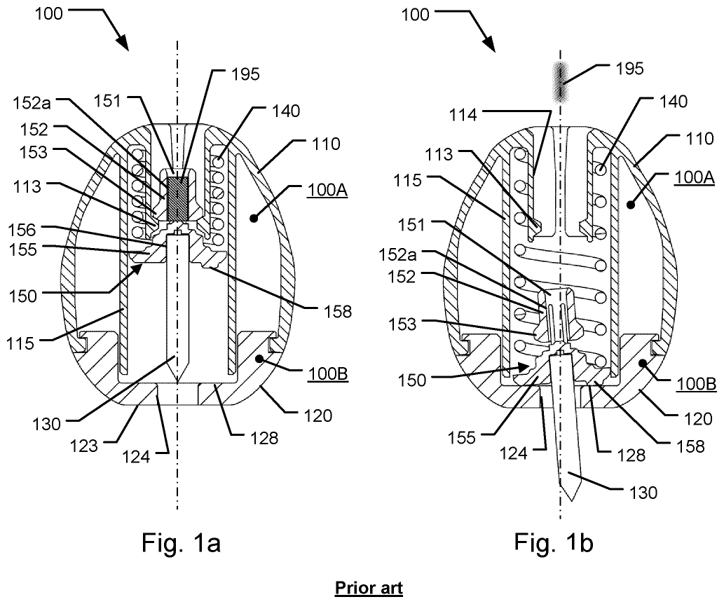

With reference to FIGS. 1a and 1b an example prior art drug delivery device 100 is shown, disclosed in WO2020/245448 A1, and having a particular actuation principle for deployment of a solid dose from a solid dose capsule device. The shown prior art capsule device 100 is intended for being ingested by a patient to allow the capsule device to enter the stomach lumen, subsequently to orient relative to the stomach wall, and finally to deploy a solid dose payload for insertion at a target location in tissue of the stomach wall. For the capsule device 100 the general principle for orienting the capsule relative to the stomach may utilize the principles disclosed in WO 2018/213600 A1.

The ingestible self-righting capsule device 100 comprises a first portion 100A having an average density, a second portion 100B having an average density different from the average density of the first portion 100A. The capsule device 100 accommodates a payload portion 130 for carrying an agent for release internally of a subject user that ingests the article. In the shown device, the average density of capsule device prior to deployment is larger than that of gastrointestinal fluid, enabling the capsule device to sink to the bottom of the stomach lumen. The outer shape of the self-righting article is a Gömböc shape, i.e. a Gömböc-type shape that, when placed on a surface in any orientation other than a single stable orientation of the shape, then the shape will tend to reorient to its single stable orientation.

The capsule device shown includes an upper (proximal) capsule part 110 which mates and attaches to a lower (distal) capsule part 120. The upper capsule part 110 and the lower capsule part 120 together forms the capsule of the device. The capsule defines an interior hollow which accommodates the payload portion 130, a hub 150 which holds and drives forward the payload portion 130, and a firing and propulsion mechanism including an actuator configured to actuate and drive forward the hub with the payload for drug delivery. The payload portion 130 is oriented along a firing axis and configured for movement along the firing axis. In the shown device, the upper and lower capsule parts 110, 120 form rotation symmetric parts which are symmetric around the firing axis. In the drawings, the device is oriented with the firing axis pointing vertically, and with the payload portion 130 pointing vertically downwards towards an exit hole 124 arranged centrally in the lower capsule part 120, the exit hole allowing the payload portion 130 to be transported through exit hole and moved outside the capsule device 100. The lower part 120 includes a tissue engaging surface 123 which is formed as a substantially flat lower outer surface surrounding the exit hole 124.

Regarding suitable materials for the capsule parts for the device shown in FIGS. 1a and 1b, the upper part may suitably be made from a low-density material, such as polycaprolactone (PCL), whereas the lower part 120 may be suitably made from a high-density material, such as 316L stainless steel.

In the shown prior art device, due to the density distribution of the entire capsule device 100, and due to the outside shape of the device, the capsule device 100 will tend to orient itself with the firing axis substantially perpendicular to the surface (e.g., a surface substantially orthogonal to the force of gravity, a surface of a tissue such as the wall of the gastrointestinal tract). Hence, the capsule device tends to orient relative to the direction of gravity so that the tissue engaging surface 123 faces vertically downward.

The interior of the upper capsule part 110 includes a sleeve shaped hub guiding structure 115 which extends concentrically with the firing axis from the upper part of the upper capsule part 110 towards a hub stop surface 128 defined by an inner bottom surface formed in the lower capsule part 120, i.e. a proximally facing stop surface. Further, in the shown device, a second sleeve shaped structure 114 extends concentrically with the firing axis and radially inside the hub guiding structure 115 from the upper capsule part 110 and downwards along the firing axis. The second sleeve shaped structure 114 serves as a retainer structure for retaining the hub 150 against the drive force emanating from a strained drive spring 140 arranged within the capsule, i.e. the drive spring serves as an actuator for driving forward the hub from a first position to a second position. In the shown device, the retainer structure has a radially inwards protruding retainer portion 113 arranged at the lower end of the retainer structure. In the shown device, the retainer portion 113 is provided as two opposed radially inwards protruding arc-shaped protrusions.

In the prior art device shown in FIGS. 1a and 1b, payload portion 130 defines a solid delivery member formed entirely or partly from a preparation comprising the therapeutic payload. In the shown device, the solid delivery member is formed as a thin cylindrical rod shaped to penetrate tissue of the lumen wall, the cylindrical rod having a tissue penetrating end and trailing end opposite the tissue penetrating end. The tissue penetrating end of the rod is pointed to facilitate easy insertion into tissue of the lumen wall whereas the trailing end, in the shown device, defines a truncated cylinder cut off by a 90-degree cut. A non-limiting example of a drug suitable for delivery by capsule device 100 is dried compressed API such as insulin.

The hub 150 comprises an upper retaining part 151 and a lower interface part 155 configured for holding the trailing end of the payload portion 130 in place. In the shown device, the interface part includes a downward open bore that receives the trailing end of the payload portion 130 in a way so that the payload portion 130 is firmly attached within the bore. The lower interface part 155 further defines an annular outer flange having a diameter slightly smaller than the diameter of the hub guiding structure 115. In the shown device, the hub 150 is movable, while being guided for axial movement by the hub guiding structure 115, from a pre-actuation configuration shown in FIG. 1a to an actuated configuration shown in FIG. 1b.

With regard to the above-mentioned drive spring 140, in capsule device 100, a helical compression spring is arranged coaxially with the firing axis. The proximal end of drive spring 140 is seated against a spring seat of upper capsule part 110, i.e. located radially between the hub guiding structure 115 and the retainer structure. The distal end of drive spring 140 is seated against a spring seat formed by a proximal surface of the flange defined by the lower interface part 155 of the hub 150. As part of assembling the capsule device 100 the drive spring 140 has been energized by axially compressing the drive spring 140 between the two spring seats. Hence, the hub is initially under load from drive spring, such as in the order of 10-30 N. Alternatives to using a compression spring for generating the drive force, other spring configurations may be used to energize the capsule device 100, such as a torsion spring, a leaf spring, a constant-force spring or similar. In further alternatives, a gas spring or a gas generator may be used.

The upper retaining part 151 of the hub 150 includes deflectable latches provided in the form of two deflectable arms 152 which extend in distal direction from the upper end of the hub towards the exit opening 124, each arm being resiliently deflectable in the radial inwards direction. The end of each deflectable arm 152 includes a blocking portion 153 protruding radially outwards from the resilient arm. In the pre-actuation configuration shown in FIG. 1a, a distal surface of each of the blocking portions 153 engage a proximal surface of a corresponding one the retainer portions 113. As the blocking portions 153 initially are located proximally to the retainer portions 113 the hub 150 cannot be moved distally past the retainer portions 113 unless the deflectable arms 152 become sufficiently deflected in the radially inwards direction.

In the pre-actuation configuration a dissolvable pellet 195 is arranged between the two deflectable arms 152 so that radial opposing surfaces of the pellet 195 engage a radially inwards facing support surface of the two deflectable arms 152. In the shown device, the pellet 195 is arranged in a compartment inside the upper capsule part 110, and a proximally arranged upper opening in upper capsule part 110 facilitates fluid exposure to the dissolvable pellet when the capsule device is submerged in a fluid. In the pre-actuation configuration shown in FIG. 1a, as the dissolvable pellet 195 assumes a non-compressible state the pellet prevents the two deflectable arms from bending inwards. However, upon exposure to a fluid, such as gastric fluid present in the stomach of a patient, the dissolvable pellet starts to dissolve. The pellet 195 is designed to become gradually dissolved so that after a predefined activation time, the pellet has been dissolved to a degree allowing the two deflectable arms 152 to become sufficiently deflected inwards enabling the blocking portions 153 of hub 150 to be moved distally past the retainer portions 113. In this condition, i.e. the actuated configuration, the hub 150 has been actuated with the load of the drive spring 140 forcing the hub 150 distally towards the exit hole 124. The hub 150 drives the payload portion 130 distally with the payload tip protruding initially from the capsule, and gradually pressing out the remaining payload portion 130. The forward movement of the payload portion 130 is halted when hub 150 bottoms out in the lower capsule part 120. This condition is depicted in FIG. 1b.

In the shown device, the interface between the retainer portions 113 and the blocking portions 153 is sloped by approximately 30° so that the deflectable arms will slide inwards when the dissolvable pellet is dissolved. The angle determines the shear forces on the pellet and to which degree the deflectable arms will tend to slide inwards when subjected to the load force. In connection with the acceleration length of the hub when fired, the optimal angle is 0°, but it requires a much higher spring force to activate such configuration. For the sloped portions, in other devices, angles other than 30° may be used.

FIG. 1b reveals that, in the shown prior art device, the hub 150 and the payload portion 130 may enter an orientation that is somewhat tilted relative to the firing axis. This effect is obtained by a tilting mechanism that tilts the hub 150 upon the hub reaching its end destination, i.e. the end of stroke position. However, the condition schematically shown in FIG. 1b is somewhat hypothetical, as it is only representative for a capsule device being fired into the open, or with the payload portion being fired into a fluid.

In situation of intended use, the payload portion 130 is inserted into tissue of the lumen wall where it will anchor generally in a direction along the firing axis. However, at the end of the drive stroke, and due to the tilting action of the hub 150, a bending torque is applied onto payload portion 130 tending to break or otherwise release the connection between payload 130 and hub 150. This effect is introduced to enable the payload portion 130 to become forcedly separated from the hub 150 to prevent that payload portion 130 becomes withdrawn from the tissue after it has been properly lodged within the tissue.

At this point the capsule device 100 has delivered the intended dose and will release relative to the deposited payload portion 130 which rests inside the tissue wall. Subsequently, the remaining parts of the capsule device will travel out through the digestive system of the user and be disposed of.

If the payload 130 where still fixedly connected to hub 150, and thus also to the remaining parts of the capsule device 100, the likelihood that payload portion would become retracted from the tissue by movements of the capsule device relative to the target location would be high.

In the shown prior art device, the tilting motion of hub 150 upon reaching the end destination is obtained by forming an eccentrically arranged protrusion 158 on the distally facing surface of interface part 155 of hub 150. As proximally facing hub stop surface 128 defined by the inner bottom surface formed in the lower capsule part 120 is planar, and oriented orthogonally to the firing axis, a tilting effect is obtained as hub 150 meets the hub stop surface 128.

For the dissolvable member discussed above, i.e. the dissolvable pellet 195 forming a dissolvable firing member, different forms and compositions may be used. Non-limiting examples include injection moulded Isomalt pellets, compressed granulate Isomalt pellets, compressed pellets made from a granulate composition of Citrate/NaHCO3, or compressed pellets made from a granulate composition of Isomalt/Citrate/NaHCO3. A non-limiting exemplary size of a dissolvable pellet is a pellet which at the time of manufacturing measures Ø1×3 mm.

In the shown prior art example of hub 150 the upper retaining part 151 is formed as a chamber wherein the dissolvable pellet 195 is received within the chamber having a tight fit. In the shown device, the central upper part of capsule device 100 includes a single opening for introducing stomach fluid within the capsule. In other devices, the capsule may include other design of fluid inlet openings such as multiple openings distributed around the capsule. In some designs, the payload portion 130 is accommodated in a chamber that is fluidly sealed from the chamber of the dissolvable pellet. Also, the exit hole 124 may include a seal preventing moisture from entering the payload portion chamber prior to firing of the capsule device 100.

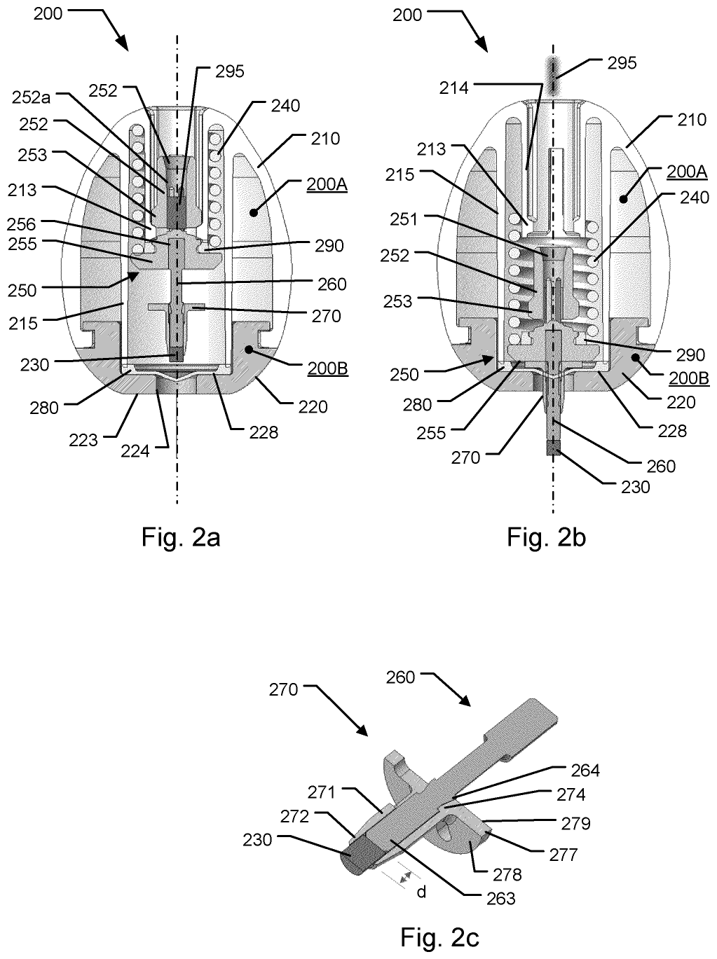

Turning now to FIGS. 2a-2c, a first embodiment of a drug delivery device in accordance with the invention is schematically shown and will be described. The first embodiment capsule device 200 is configured as an ingestible self-righting capsule device comprising a first upper portion having a first density, a second lower portion having a second density different from the first density of the upper portion. The capsule device 200 accommodates a payload portion 230, forming a tissue penetrating member, for carrying an agent for release internally of a subject user that ingests the article. In the shown device, the density of the capsule device, at least in the state prior to deployment, is greater than that of gastrointestinal fluid, enabling the capsule device to sink to the bottom of the stomach lumen. In the shown embodiment, the capsule device defines a monostatic body. The outer shape of the self-righting article is a Gömböc shape, i.e. a Gömböc-type shape that, when placed on a surface in any orientation other than a single stable orientation of the shape, will tend to reorient to its single stable orientation. The density distribution of the first upper portion and the second lower portion is provided so that the capsule device 200 quickly seeks towards entering a stable orientation wherein a central longitudinal axis of the capsule body is oriented vertically or generally vertical so that the tissue penetrating member 230 aligned with a trigger axis (coaxial with the longitudinal axis) assumes an orientation generally perpendicular to the portion of the tissue wall that supports the lower portion of the capsule.

In accordance herewith, the first embodiment capsule device 200 includes an upper (proximal) capsule part 210 which mates and attaches to a lower (distal) capsule part 220. The two parts 210 and 220 are interconnected by means of a snap fit connection. In other embodiments, the two parts 210 and 220 are joined by other mounting methods such as by means of a threaded connection or a bayonet connection. The upper capsule part 210 and the lower capsule part 220 together forms a shell part of the capsule device 200, i.e. the exterior of the capsule housing. The capsule defines an interior hollow which accommodates the payload portion 230, an assembly of components which in combination form a push member/hub 250 which holds and drives forward the payload portion 230, and an actuation arrangement configured to actuate and drive forward the push member/hub 250 carrying with it the payload portion 230 for drug delivery. The payload portion 230 is initially arranged within a sealed payload chamber and oriented along said trigger axis. The payload portion 230 is configured, upon triggering of the capsule device, for movement along the trigger axis. In the shown device, the exterior portions of upper and lower capsule parts 210, 220 form generally rotation symmetric parts arranged with their axis of symmetry coaxially with the trigger axis. In the drawings, the device is oriented with the trigger axis pointing vertically, and with the payload portion 230 pointing vertically downwards towards an exit hole 224 arranged centrally in the lower capsule part 220, the exit hole allowing the payload portion 230 to be transported through exit hole and moved outside the capsule device 200. As shown, the lower part 220 includes a tissue engaging surface 223 which is formed as a substantially flat lower outer surface surrounding the exit hole 224.

With the capsule device 200 in the pre-actuation configuration, the payload portion 230 assumes the position shown in FIG. 1a within the sealed payload chamber so that the distal end surface of the payload portion is axially separated, i.e. by a separating distance, from the distal surface encircling the exit opening thereby enabling the payload portion to be accelerated towards the exit opening and further to the surface of the tissue by an acceleration stroke corresponding to the separating distance. In this embodiment the separating distance is selected in the order of 2-3 mm.

Regarding exemplary materials for the capsule parts for the capsule device 200, the upper part 210 may suitably be made from a low-density material, such as polycaprolactone (PCL) or Polyether ether ketone (PEEK), whereas the lower part 220 may be suitably made from a high-density material, such as 316L stainless steel.

The interior of the upper capsule part 210 includes a first sleeve shaped structure 215 extending concentrically with the trigger axis and providing a radially inwards facing cylindrical hub guiding structure which extends concentrically with the trigger axis from the upper part of the upper capsule part 210 towards the lower capsule part 220. Further, in the first embodiment, a second sleeve shaped structure 214 extends concentrically with the trigger axis and radially inside the hub guiding structure from the upper capsule part 210 and downwards along the trigger axis. The second sleeve shaped structure serves as a retainer structure and includes retainer portions 213 for retaining the hub 250 against the drive force emanating from strained compression drive spring 240 arranged within the capsule, i.e. the drive spring serves as an energy source for driving forward the hub from a first position to a second position.

The upper retaining part 251 of the hub 250 includes deflectable latches provided in the form of two deflectable arms 252 which extend in distal direction from the upper end of the hub, each arm being resiliently deflectable in the radial inwards direction. The end of each deflectable arm 252 includes a blocking portion 253 protruding radially outwards from the resilient arm. In the pre-actuation configuration shown in FIG. 1a, a distal facing surface of each of the blocking portions 253 engage a proximal facing surface of a corresponding one the retainer portions 213. As the blocking portions 253 initially are located proximally to the retainer portions 213 the hub 250 cannot be moved distally past the retainer portions 213 unless the deflectable arms 252 become sufficiently deflected in the radially inwards direction.

In the pre-actuation configuration a dissolvable pellet serves as a dissolvable latch support 295 being arranged between the two deflectable arms 252 so that radial opposing surfaces of the dissolvable latch support 295 engage a radially inwards facing support surface of the two deflectable arms 252. In the shown device, the dissolvable latch support 295 is arranged in a compartment inside the upper capsule part 210, and a proximally arranged upper opening in upper capsule part 210 facilitates fluid exposure to the dissolvable latch support when the capsule device is submerged in a fluid. In the pre-actuation configuration shown in FIG. 1a, as the dissolvable latch support 295 assumes a non-compressible state the pellet prevents the two deflectable arms from bending inwards. However, upon exposure to a fluid, such as gastric fluid present in the stomach of a patient, the dissolvable latch support starts to dissolve. The dissolvable latch support 295 is designed to become gradually dissolved so that after a predefined activation time, the pellet has been dissolved to a degree allowing the two deflectable arms 252 to become sufficiently deflected inwards enabling the blocking portions 253 of hub 250 to be moved distally past the retainer portions 213. In this condition, i.e. the actuating configuration, the hub 250 has been actuated with the load of the drive spring 240 forcing the hub 250 distally towards the exit hole 224. The hub 250 drives the payload portion 230 distally with the payload protruding initially from the capsule, and gradually pressing the payload portion 230 deeper into the supporting tissue. The forward movement of the payload portion 230 is halted when hub 250 bottoms out relative to the lower capsule part 220. This condition is depicted in FIG. 1b.

In the shown device, the interface between the retainer portions 213 and the blocking portions 253 is sloped by approximately 30° so that the deflectable arms will slide inwards when the dissolvable pellet is dissolved. The angle determines the shear forces on the pellet and to which degree the deflectable arms will tend to slide inwards when subjected to the load force. In connection with the acceleration length of the hub when fired, the optimal angle is 0°, but it requires a much higher spring force to activate such configuration. For the sloped portions, in other devices, angles other than 30° may be used.

The first embodiment capsule device 200 additionally comprises a pair of sealing elements 280, 290 for maintaining the payload portion 230 fluidically isolated from the environment external to capsule device 200 prior to actuation. In the shown embodiment, an upper sealing element 290 formed as a ring of soft pliable material, such an elastomeric material, is inserted between the lowermost annular surface second sleeve shaped structure 214 and an annular proximal facing flange surface of the hub 250.

The further sealing element, i.e. the lower sealing element 280, forms a fluidic gate configured to maintain the exit hole 224 fluidically blocked prior to actuation. In the shown embodiment, the sealing element 280 comprises an elastomeric seal member having a generally disc shaped form. An outer periphery of the sealing element 280 is mounted below the lowermost annular surface of hub guiding structure 215 and clamped above an annular proximally facing surface of lower capsule part 220. As disclosed in US 2020/0129441 A1 the central area of the sealing element 280 may comprise a fluidic gate formed to provide a self-sealing valve, such as formed by one or more thin cuts (e.g., one or more thin slits) that extend partially or completely through a thickness of the fluidic gate.

The sealing elements 280 and 290 thus cooperate to form a compartment internally in capsule device 200 that serves, prior to actuation, to maintain the payload portion 230 fluidically isolated from biological fluid externally to capsule device 200 but allows the payload portion to penetrate easily through sealing element 280 at the time of actuation for payload delivery into tissue.

In the shown embodiment, the hub 250 comprises an upper retaining part 251 and a lower interface part 255. The lower interface part 255 defines an annular outer flange having a diameter slightly smaller than the diameter of the hub guiding structure 215. The distal facing end surface of lower interface part 255 includes a bore that extends proximally at the centre of lower interface part 255. A proximally facing bore 256 is formed in distal facing surface of lower interface part 255. This bore 256 is configured to fixedly receive the most proximal portion of a push rod 260 which serves to transmit distal forces from hub 250 to a payload portion 230.

In this embodiment the payload portion 230 is formed as a small cylindrical tablet with outer dimensions of approximately ø0.8×0.82 mm height. Other dimensions and different shapes of the payload portions may be used in alternative embodiments. For example, the shown distal facing surface of payload portion 230 may be formed differently, such as being provided with a pointed distal spike portion pointing towards the centre of exit hole 224.

Referring mainly to FIG. 2c, in the shown embodiment, the payload portion 230 is not directly attached to the push rod 260 but will either abut from the outset or will engage push rod 260 during a part of the displacement push rod 260 experiences during actuation. Instead of being directly held by push rod 260, a holder portion provided as a holder sleeve 270 encircles both the push rod 260 and a proximal portion of the payload and which in the pre-actuation configuration attaches both to push rod 260 and to payload portion 230. The holder sleeve 270 is formed with a distal portion 271 formed as a generally cylindrical sleeve with an inner diameter generally corresponding to an outer diameter of a distal portion 263 of push rod 260. The inner diameter of the cylindrical sleeve also generally corresponds to the outer diameter of the payload portion 230. The holder sleeve 270 is further formed so that, at the proximal end of the cylindrical sleeve, a circular flange 277 extends radially outwards from an outer wall of the cylindrical sleeve. The circular flange 277 of the holder sleeve 270 defines a distally facing stop surface 278 configured for cooperation with the hub stop surface 228 via sealing element 280 and a proximal facing surface 279 configured for cooperation with the lower interface part 255 of hub 250.

In the embodiment shown, the dimensions of the inner surface of the cylindrical sleeve of holder sleeve 270 is chosen so that a frictional fit between a distal end 272 of the holder sleeve 270 and the payload portion 230 is provided. Also, in the shown embodiment, cooperating releasable snap geometries are formed between the push rod 260 and the holder sleeve 270. In FIG. 2c, the snap geometries can be seen as an inner snap geometry 274 formed on holder sleeve 270 and an outer snap geometry 264 formed on push rod 260. In the shown embodiment, the holder sleeve 270 is so dimensioned that the holder sleeve distal portion 272 overlaps axially with the payload portion 230 in a way so that approximately half of the axial height of the payload portion is gripped by holder sleeve distal portion 272, i.e. the distance denoted “d” in FIG. 2c.

Non-limiting exemplary materials for the holder sleeve 270 and the push rod 260 may be selected as a relatively resilient polymeric material such as a material formed from PEEK. Alternative materials for holder sleeve 270 may be formed by an elastomeric material, such as silicone rubber.

Referring to FIG. 2a again, the push rod 260 is seen suspended from lower part interface 255 of hub 250. Although these two components in the shown embodiment are formed as two independent parts that are joined during assembly, alternative embodiments may provide corresponding features by means of a unitary component. The system is in this embodiment designed so that, in the pre-actuation configuration, the distal face of payload portion 230 is disposed approximately 2 mm from the tissue engaging surface 223, meaning that the assembly formed by the hub 250, the push rod 260, the holder sleeve 270 and the payload portion 230, is moved together by an acceleration stroke of approximately 2 mm before the payload portion engages adjacent tissue situated below exit hole 224.

In the shown embodiment, the push rod 260 is further dimensioned to provide an insertion depth of the distal portion of payload portion 230 in the order of approximately 4 mm relative to the tissue engaging surface 223. In the shown embodiment, the lower interface part 255 of hub 250 forms a distal facing blocking surface which is configured for abutting contact with proximal facing surface 279 of holder sleeve 270. The distal facing surface 278 of holder sleeve 270 is configured for abutting contact with a proximal facing surface of sealing element 280.

Turning now to the operation of the first embodiment capsule device 200, reference is initially made to FIG. 2a which shows the capsule in an initial state representing the state the capsule device assumes during storage or just after ingestion. In this state, the hub 250 assumes the pre-actuation configuration where the two deflectable arms 252 are maintained in the shown position by engagement with the radially outwards facing surface of the dissolvable latch support 295 engaging the radially inwards facing surface 252a of the deflectable arms 252. As a result, the blocking portions 253 engage a proximal surface of a respective one the retainer portions 213 preventing the deflectable arms from sliding relative to the retainer portions. As the deflectable arms 252 are trapped proximally to retainer portions 213 the hub 250 cannot be moved distally even though the drive spring 240 exerts its full compressive load onto the hub 250.

The upper sealing element 290 engages the lowermost annular surface of second sleeve shaped structure 214 as well as the annular proximal facing flange surface of the hub 250 to keep this interface fluid tight. Also, the lower sealing element 280 keeps the exit hole 224 fluid tight.

After ingestion of capsule device 200, the capsule device quickly sinks to the bottom of the stomach. Upon being supported by the stomach wall, due to the self-righting ability of the capsule device, the capsule device will quickly reorient to have its tissue interfacing surface 223 engaging the tissue stomach wall with the trigger axis of the capsule device oriented virtually vertical, i.e. with the payload portion 230 and the push rod 260 pointing downwards. Dissolvement of dissolvable latch support 295 has begun due to exposure to gastric fluid. This is represented in FIG. 1b in connection with reference 295. The support from dissolvable latch support 295 against the deflectable arms 252 will cease at a specific time after swallowing. The load of the drive spring 240 will cause the deflectable arms 252 to be gradually deflected radially inwards thereby allowing the arms to slide off from engagement with the retainer portions 113. At some point in time the deflectable arms 252 will reach their radially collapsed position where after the hub 250 with the payload, the push rod 260, the holder sleeve 270 and the payload portion 230 will become released from the capsule housing. This state corresponds to the actuating configuration (not shown).

As drive spring 240 exerts load onto hub 250, the push rod 260, the holder sleeve 270 and the payload portion 230 are caused to travel unhindered towards the exit hole 224 as slaved with the hub 250, with the payload portion penetrating the lower sealing element 280 and further into mucosal tissue at the target location. During the distal movement of the said components, the holder sleeve distally facing stop surface 278 will enter into engaging abutment with the lower sealing element 280 and will be prevented from moving further distally. However, in this state, the remaining load of drive spring 240 will still urge the push rod 260 further distally. As the snap engagement 264/274 defines a relatively weak force transmission, the snap engagement will release causing the push rod 260 to be moved further distally carrying with it the payload portion 230 for distal sliding movement relative to the holder sleeve 270. Soon after, the payload portion will be pushed by the push rod 260 to the target depth in tissue which occurs when the distal facing blocking surface of lower interface part 255 of hub 250 engages in abutting contact with proximal facing surface 279 of holder sleeve 270. This blocks hub 250 from moving further distally. This state is depicted in FIG. 2b, note however that the spring is only schematically shown as deployed. In real-life settings the spring would have expanded between a proximal spring seat and the lower interface portion 255.

In the shown embodiment, the target insertion depth of the distal face of the payload portion is designed to be approximately 4 mm into mucosal tissue. For stomach wall deployment, further exemplary insertion depths may be selected between 3 to 7 mm, such as between 4 to 6 mm or such as between 4.5 mm and 5.5 mm.

In situation of intended use, the payload portion 230 is inserted into tissue of the lumen wall where it will anchor generally in a direction along the actuation axis. As discussed in this disclosure, depending on the specific design of the capsule device, the payload portion 230 will be released actively from the remaining parts of the capsule at the end of the insertion stroke. When the capsule device 200 has delivered the intended dose the capsule will release relative to the deposited payload portion 230 which remains inside the tissue wall for release of therapeutic agent into the blood stream of the subject.

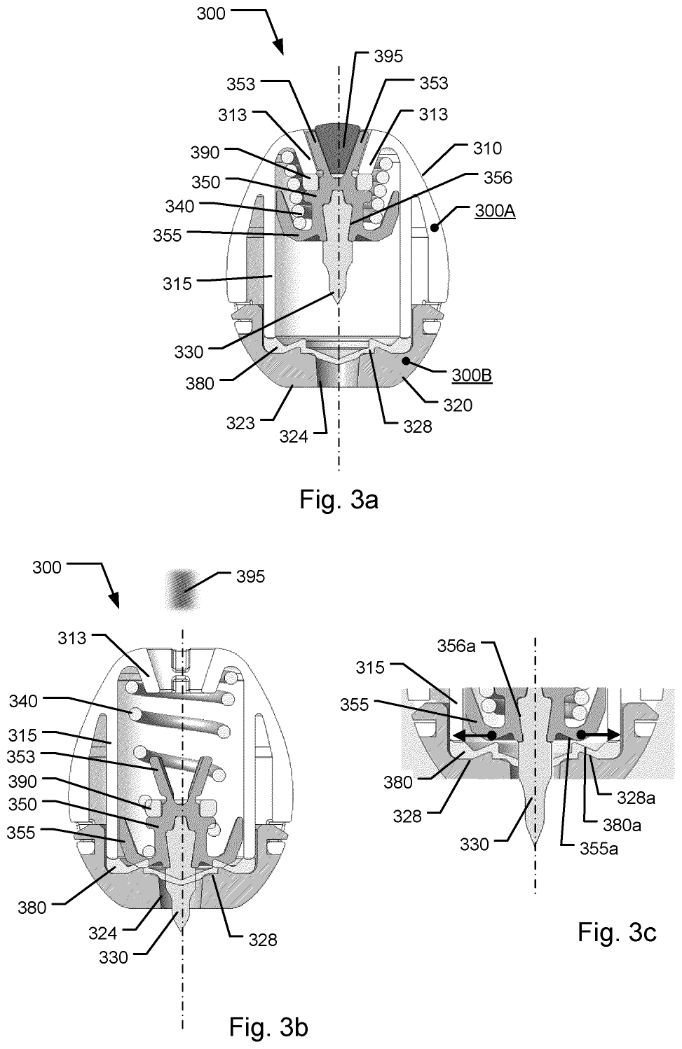

Turning next to a second embodiment of a capsule 300 reference will be made to FIGS. 3a-3c. The capsule device 300 shares many constructional features with the corresponding features of the first embodiment capsule device 200 described above, and the general principle of operation is the same. However, the payload holding portion and release function has been modified. Also parts of the actuating mechanism including the trigger release design has been modified.

Whereas, for the first embodiment where the payload holding portion and release function is provided by three components, i.e. hub 250, push rod 260 and holder sleeve 270, which includes the specified relative movement for release of payload 230, the second embodiment includes a similar functionality in a single unitary component, i.e. hub 350. To provide this, the hub 350 is formed from a largely resilient material which releasably grips the payload portion 330 at interface surface 356a until the hub 350 is deformed to release said grip, said release being caused by interaction between the hub and the lower interior surface of the capsule housing.

Referring to FIG. 3a, which show the capsule 300 in the pre-actuation configuration, the overall design of the actuation mechanism is somewhat modified for the second embodiment device 300 as compared with the first embodiment device 200, i.e. with a dissolvable latch support 395 formed as a cone shaped element to support resilient arms provided in a v-shaped configuration. The hub 350 comprises an upper retaining part configured for releasably retaining the hub relative to a retainer structure 313 of the capsule housing and a lower interface part 355 of hub 350 configured for holding the trailing end of the payload portion 330 in place. In the shown embodiment, the lower interface part 355 includes a downward open bore 356 that receives the trailing end of the payload portion 330 in a way so that the payload portion 130 is initially firmly retained within the bore. The lower interface part 355 further defines an annular spring seat for the drive spring 340. Apart from this the distal facing end surface of lower interface part 355 comprises a set of inclined surfaces 355a which will be discussed further below.

Each resilient arm, i.e. latch arm 353, is resiliently movable in the radial inwards direction by a swiveling movement relative to the upper retaining part of hub 350. The latch arms 353 each defines a radially outwards facing latch surface configured to engage with respective portions of a conical retainer surface portion 313 in a latching engagement. Each of the latch arms 353 further includes a radially inwards facing latch surface configured for matingly cooperating with centrally disposed conically shaped dissolvable latch support 395.

Also for the second embodiment capsule device 300, the capsule comprises a pair of sealing elements 380, 390 for maintaining the payload portion 330 fluidically isolated from the environment external to capsule device 300 prior to actuation. In the shown embodiment, an upper sealing element 390 formed as a ring of soft pliable material, such an elastomeric material, is inserted between the lowermost annular surface of structure 313 and an annular proximal facing flange surface of the hub 350.

The hub stop surface 328 of lower capsule part 320 is in this embodiment formed with inclined surfaces which serve as releasing surfaces for hub 350 causing release of payload portion 330 from hub 350 upon cooperation of hub 350 with the hub stop surface 328, i.e. in a position slightly prior to the end of stroke position. Also the sealing element 380 includes similar inclined surfaces as hub stop surface 328.

FIG. 3b shows schematically the capsule device 300 in the actuated configuration, wherein the hub 350 has been forced fully distally to abut with the hub stop surface 238. However, it shall be noted that, in FIG. 3b, cooperating inclined surfaces 380a of the hub sealing element 380 and the inclined surfaces 355a of hub 350 are incorrectly shown as interposed elements which does not accurately reflect the real-life situation for the impacted elements. The following figure serves to more properly describe the system before the end of stroke impact between hub 350 and hub stop surface 328.

FIG. 3c shows the capsule device 300 in a state just prior to the actuated state shown if FIG. 3b. In this illustration in FIG. 3c it is seen how inclined surfaces 328a/380a, each having a surface normal pointing proximally and radially outwards, are structured to cooperate with an inclined surface 355a (of hub 350) having a surface normal pointing distally and radially inwards. The said pair of cooperating surfaces are provided as a multitude of pairs of cooperating surfaces distributed around the firing axis, i.e. arranged symmetrically relative to the firing axis. When the pairs of cooperating surfaces engage, i.e. near the end of stroke position for hub 350 inside capsule 300, the inclined surfaces 380a induce a radially outward directed force, indicated by arrows, on lower interface part 355 causing these portions of lower interface part 355 to be deformed radially outwards. This in turn causes the material portions at the interface surface 356a to be moved radially outwards resulting in bore 356 becoming widened radially and in this way releasing the grip of payload portion 330.

In the shown embodiment, the capsule may be so designed that, as the release of grip on payload portion 330 occurs, i.e., shortly before the hub 350 becomes arrested relative to hub stop surface 328, the inertia of payload portion 330 will result in the payload becoming shot a little further relative to the release position represented by the actuated configuration, and the payload portion will be inserted into tissue of the lumen wall at the desired insertion depth. However, such “throw” effect is only optional and may be omitted if desired.

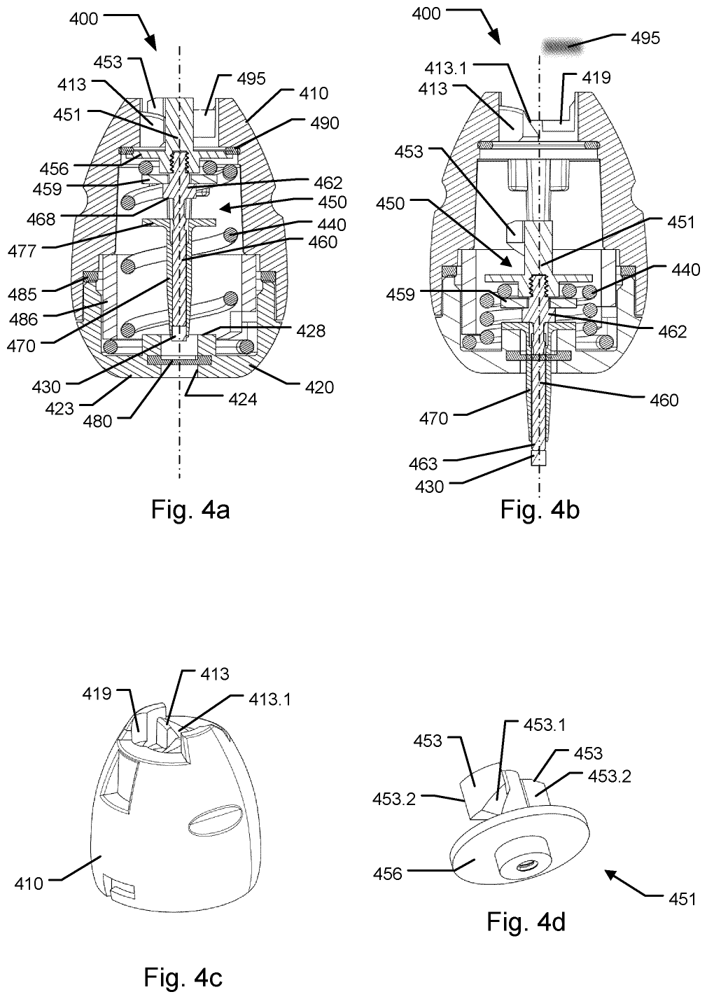

Referring to FIGS. 4a-4d, a third embodiment drug delivery device 400 in accordance with the invention is shown and will be described in the following.

With regard to the self-righting ability and the principle for deployment of the payload into tissue, the third embodiment capsule device 400 generally corresponds to the overall design of the first example capsule device 200, but the actuator principle and the way the hub is released from the capsule housing is different. In the shown embodiment, the assembly made up of hub lower part 460, holder sleeve 470 and payload portion 430 generally correspond to the design shown in FIG. 2c.

FIGS. 4a and 4b are side views of the third embodiment capsule device 400 in the pre-actuation configuration and the actuated configuration, respectively. The drive spring 440 in this design is provided as a tension spring having a constant outer diameter along a major part of its axial extension. An enlarged diameter winding at the distal end of drive spring 440 aids in mounting the distal end of the drive spring relative to the capsule housing. A spacer element 486 is inserted into a cylindrical bore of the upper capsule part 410. The enlarged diameter winding of spring 440 and the spacer element 486 are clamped axially between a distally facing surface of upper capsule part 410 and a proximally facing surface of the lower capsule part 420. By incorporating a tension spring, the spring interface towards the hub can be placed far away from the bottom of the device, maximizing both spring force and total stroke.

The proximal end of drive spring 440 includes a reduced diameter winding which aids in coupling to the hub 450. In the shown design the reduced diameter winding is clamped axially between a proximal flange 456 and a distal flange 459. The distal flange 59 performs as a washer and is mounted rotatably relative to hub upper part 451 and hub lower part 462 so that torsional forces incurred by rotational movement of hub upper part 451 and hub lower part 462 are not transferred to the drive spring 440.

In the pre-actuation configuration, a hub lock geometry and a housing lock geometry engage each other so as to maintain the hub 450 positioned in an initial first position against the axial tension load exerted by drive spring 440. The hub lock geometry defines a hub retaining surface whereas the housing lock geometry defines a housing retaining surface. In this embodiment the hub retaining surface and the housing retaining surface are provided as ramped geometries 453.1 and 413.1, these to be described further below.

The third embodiment again comprises a lower sealing element 480 to seal off the exit opening 424 and an upper sealing element 490, arranged between a flange 460 on the hub upper part 451 and a distal facing rim surface formed in upper capsule part 410, to seal the payload chamber at this interface. In addition an intermediate seal 485 is arranged at the interface between the upper capsule part 410 and the lower capsule part 420.

The upper capsule part 410 of capsule device 400 is depicted in FIG. 4c. Upper capsule part 410 this time includes two pellet receiving pockets 419 which are symmetrically disposed around the axis. Only one of the pellet receiving pockets 419 is designated for being used during assembly but the symmetry enables the mounting of components in either of two orientations. Encircling a central axial passage formed within upper capsule part 410 are two ramp shaped geometries 413.1 formed on respective housing lock geometries 413, each ramp forming a helically curved arc surface spanning an angle of approximately 80 degrees and oriented so that the curved surface extends in a distal-clockwise direction (as viewed from above, see top views 2f and 2g). Each of the curved surfaces 413.1 are designated for use with correspondingly curved surfaces 453.1 of hub lock geometries formed as wings 453 by hub upper part 451.

As shown in FIG. 4d the hub upper part 451 comprises the flange 456 and a cylindrical top part extending in the proximal direction from flange 456. The wings 453 are disposed on the cylindrical top part so that the wings protrude radially outwards in a radially opposed manner. Each wing defines an arc which is 60 degrees wide in the rotational direction around the axis. The central axial passage formed by upper capsule part 410 allows passage of the wings 453 of hub upper part 451 but only when the hub upper part 451 is oriented so that the wings 453 clear the helically curved arc surfaces 413.1. With the capsule device 400 assuming the pre-triggering configuration, the curved surfaces 453.1 of wings 453 intimately engages the ramp shaped geometries 413.1 of upper capsule part 410 so that the hub upper part 451 rests on these inclined surfaces while the tension force of the strained drive spring 440 urges the hub upper part 451 in the distal direction.

The tension force from drive spring 440 induces a torsional force on the hub upper part 451 in clockwise direction (seen from above) due to the inclined surfaces 413.1 and 453.1. However, as shown in FIG. 4a, a dissolvable pellet 495 is arranged in one of the pellet receiving pockets 419 and a radial extending surface 453.2 of one of the wings 453 engages a side surface of the dissolvable pellet 495 thereby preventing the hub upper part 451 from sliding distally on the inclined surfaces 413.1.

Turning now to the operation of the third embodiment, after ingestion of capsule device 400, the capsule device quickly sinks to the bottom of the stomach. Upon being supported by the stomach wall, and due to the self-righting ability of the capsule device, the capsule device will quickly reorient to have its tissue interfacing surface 423 engaging tissue of the stomach wall with the trigger axis of the capsule device oriented virtually vertical, i.e. with the payload portion 430 and the push rod 460 pointing downwards. Dissolution of dissolvable pellet 495 has begun due to exposure to gastric fluid. This is schematically represented in FIG. 4b in connection with reference 495. The support from dissolvable pellet 495 on the radial extending surface 453.2 will at some point cease allowing the wings 453 of hub upper part to slide distally along the curved arc surfaces 413.1. Subsequent to full dissolution of pellet 495, or subsequent to the pellet 495 becoming dissolved to a degree so that the pellet 495 will be pushed away from its blocking position within pocket 419, and after approximately 60 degrees of turning movement of the hub upper part 451, the inclined surfaces 453.1 of wings 453 will slide off the curved arc surfaces 413.1 and be free to move further distally within the capsule device through the central axial passage. As shown in FIG. 4b, the distal movement of the hub 450 is halted when the hub lower part 462, and more specifically the distal end surface 468 bottoms out in the capsule interior. In this position the drive spring 440 has pulled the hub 450 into an axial position wherein the flange 477 of holder sleeve 470 is clamped between distal end surface 468 and hub stop surface 428. In this state the capsule device 400 assumes its deployed configuration.

The subassembly of push rod 460, holder sleeve 470 and payload portion 430 is largely similar to the corresponding elements of the first embodiment, as shown in FIG. 2c. However, for the first embodiment, the circular flange 277 of the holder sleeve 270 defines a distally facing stop surface 278 configured for cooperation with the hub stop surface 228 indirectly via sealing element 280 to define the end of stroke position for the push rod 260. In such embodiment the housing stop geometry is provided by means of the proximal facing surface of the sealing element 280, this element being fixedly associated with the capsule housing. Opposed to this, in the third embodiment capsule device 400, the distal end surface 468 of hub lower part 462 engages directly with the hub stop surface 428, the latter defining a housing stop geometry which axially stops the holder portion 460 from further movement.

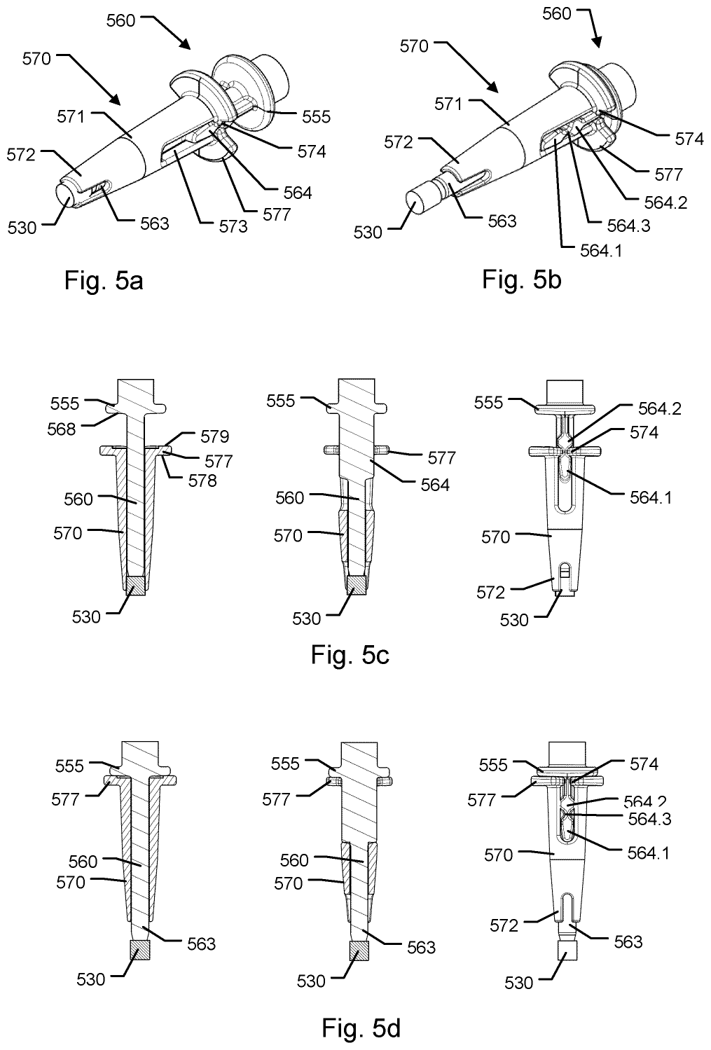

Turning now to FIGS. 5a through 5d, these figures are different representations of a subassembly of a fourth embodiment capsule device, the subassembly consisting of a push rod 560, a holder sleeve 570 and a payload portion 570, these components generally corresponding to the push rod 260, holder sleeve 270 and payload portion 230 of the first embodiment. In the fourth embodiment holder portion 570 is again provided as a generally sleeve shaped element. The sleeve is formed with a distal portion 571 formed as a generally cylindrical sleeve with an inner diameter generally corresponding to an outer diameter of a distal portion 563 of pushing portion (push rod 560). In the shown embodiment, the distal portion 571 of the holder sleeve is slightly conically shaped so as to taper into a smaller diameter towards its distal end face. This may facilitate easy insertion/incision of the holder sleeve 570 into tissue at a target location of a lumen wall.

The distal portion 571, due to axially extending slots which extend a minor distance proximally from the most distal portion of the holder sleeve 570, forms a number of part cylindrical shells 572, in the shown embodiment formed as two half shells. Due to the presence of the slots the half shells 572 are somewhat resilient in the radial direction and configured for being bent slightly radially outwards. Compared to the inner diameter of the half shells 572, i.e., when these assume an unbiased state, the payload portion 530 is somewhat larger in diameter. Hence, when the payload portion 530 is coaxially positioned radially between the half shells 572 the half shells are flexed radially outwards and a frictional engaging grip is provided by the holder sleeve 570 thereby facilitating a releasable axial retaining engagement of payload portion 530 relative to the holder sleeve 570. It is to be noted that other means of providing radially resilience of the distal portion 571 may be provided in other designs, such as providing different numbers of slots and different geometries of shells or similar elements, such as axially extending arms or circumferentially extending arms. Such arms will typically be defined by slots separating the arms from the remaining portions of a holder portion, e.g. formed by slots extending in a non-axial direction, such as transverse to the axis and/or with a spiral shape.

In still other embodiments the holder portion may, instead of being sleeve-shaped, be formed alternatively, such as being formed as plurality of axially extending arms that are configured to provide a radially resilient grip, e.g., similar to forceps, which releasably hold the payload portion at a radially outwards surface and/or a distal facing surface.

In the fourth embodiment, at the proximal end of the holder sleeve 570, a radially outwards extending flange 577 is disposed. The proximal part of the sleeve further comprises two axially extending through going slots 573 that cuts distally into the holder sleeve axially across the flange 577. This enables two proximal part cylindrical shells to be defined which both provide some resilience against being moved radially outwards.

The push rod 560 includes at its proximal portion a circular flange 555 which provides a distally facing stop surface 568 for abutting engagement with a proximally facing surface of flange 577 of holder sleeve 570. Along opposed side portions of the push rod 560 a pair of diametrically opposed snap protrusions 564 extend radially outwards. Each snap protrusion 564 is configured to be received in a respective one of the slots 573 of the holder sleeve. Each snap protrusion is formed as an elongated axially extending ridge having a variable width profile in the tangential direction, with a relatively wide first distal protrusion 564.1, a relatively wide second proximal protrusion 564.2 and with a narrow bridge portion 564.3 connecting the distal protrusion 564.1 and the proximal protrusion 564.2.

The through going slots 573 are, at the axial location of the flange 577, provided with a respective snap opening 574, each snap opening configured to cooperate with the profiled ridges of protrusions 564.1, 564.2, 564.3. In the view shown in FIG. 5a, the push rod 560 assumes an initial position, corresponding to the pre-actuating configuration, wherein the bridge portion 564.3 is received within a snap opening 574 and the two proximal part cylindrical shells are in a relaxed state. The holder sleeve 570 is prevented from unintentionally moving proximally and distally relative to the push rod 560 due to the presence of the wide first distal protrusion 564.1 being disposed distally to the snap opening 574 and the wide second proximal protrusion 564.2 disposed proximally to the snap opening.

The holder sleeve 570 defines a distal facing end surface. When the holder portion 570 holds the payload portion 530 in axial retaining engagement, as shown in FIG. 5a, the distal facing end surface of the holder sleeve 570 partially encircles the payload portion 530 while the payload portion extends distally from the distal facing end surface of the holder portion. Hence, when the push rod 560 starts to advance, the holder portion and the payload portion are carried along distally and the payload portion 530 acts as a tissue penetrating member forcing its way through tissue for the entire tissue insertion procedure. Further details for the assembly in the pre-actuation configuration can be viewed in the side views shown in FIG. 5c.

FIG. 5b shows the assembly in the actuated configuration wherein the force of the energy source of the capsule device has been triggered for release and the load of the energy source has moved the push rod 560 the entire way towards the housing stop. As the flange 577 will be arrested during the actuation procedure the push rod will exert continued force on the connection between the wide second proximal protrusion 564.2 onto the snap opening 574 acting to let the protrusion 564.2 pass the snap opening, due to the two proximal part cylindrical shells of the holder sleeve being forced radially away from each other. This will enable the push rod 560 to displace distally relative to the holder sleeve 570 and will result in the push rod forcing the payload portion 530 distally relative to the holder sleeve 570 thus releasing the axial retaining engagement between the two. The profiled ridges of protrusions 564.1, 564.2, 564.3 will enter further into the slots 573 until state wherein the circular flange 555 of push rod 560 enters into abutment with the flange 577. The payload portion will be disposed at the target location at the desired depth of insertion into tissue for proper drug deposition. It is noted that the payload portion 530 has been axially separated a distance from the distal facing surface of the holder sleeve 570. Further details for the assembly in the actuated configuration can be viewed in the side views shown in FIG. 5d.

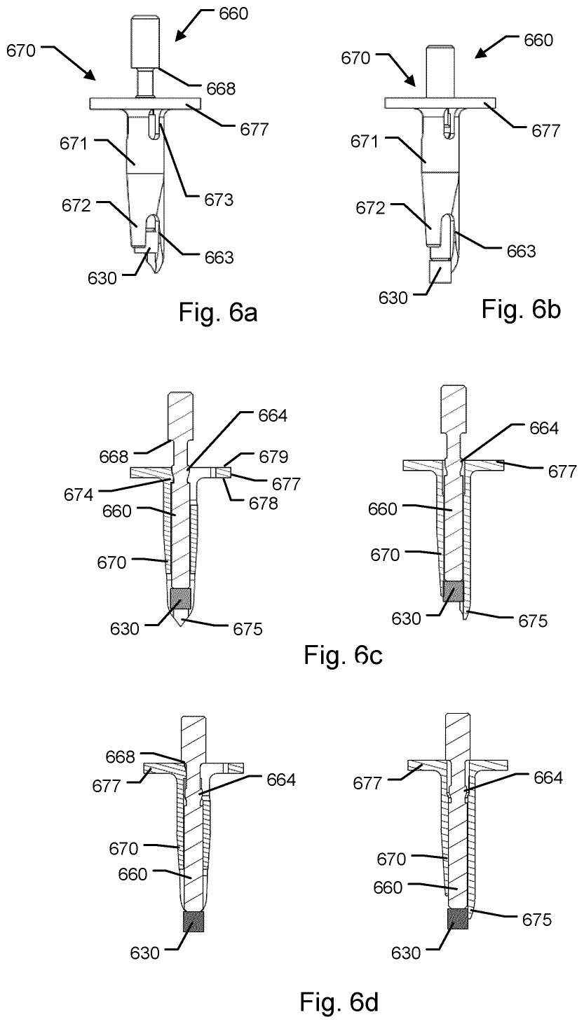

Turning now to FIGS. 6a through 6d, these figures are different representations of a subassembly of a fifth embodiment capsule device, the subassembly consisting of a push rod 660, a holder sleeve 670 and a payload portion 670, these components generally corresponding to the push rod 260, holder sleeve 270 and payload portion 230 of the first embodiment.

In comparison with the first embodiment, the fifth embodiment mainly differ in that a piercing element 675 extends distally relative to the distal portion of the holder sleeve 670. In accordance with different embodiments, the piercing element 675 may protrude even further distally than the distal end of the payload portion 630, such as by protruding 0.5 mm to 3 mm further than the payload portion, i.e., with the assembly assuming the pre-actuation configuration.

The piercing element 675 may be provided with one or more sharp edges and/or pointed geometries to facilitate puncturing of the tissue penetrating element 675 and further aiding in improved penetration of the payload portion 630 as it enters into the shallow portion of the mucosa. The optimized geometry or geometries provide a cutting surface at their tips to initiate tissue cracking and therefore result in a reduction in penetration force for inserting the payload portion. Hence, for some embodiments, the piercing element 675 only pierces tissue during the initial insertion into tissue whereas the payload portion penetrates more deeply into mucosa when the push rod 660 is being displaced relative to the holder sleeve 670.

In further embodiments, the holder sleeve may include a plurality of piercing elements that extends distally from the payload portion while the remaining parts of the distal sleeve encircles the payload portion, such as approximately axially midways on the payload portion.

For some embodiments, such as different embodiments according to the invention, the presence of small, sharp, protruding elements in an oral device, could potentially pose a risk of scratching, ripping, puncturing etc of the tissue of especially the intestine during transfer through the gastric system.

Utilizing softening/swellable/degradable materials for manufacturing of any components that protrude from the device after drug deposition, such as the holder sleeve and the push rod, will eliminate the risk of harm to the gastric intestinal tissue, as the mechanical integrity of the protruding elements are lowered.

The materials come into contact with the biological environment of the gastric system i.e. either the blood during insertion of the API, gastric contents (gastric fluids), mucus in the stomach, intestinal fluids etc and the change of physical properties are initiated. The change of properties could include but are not limited to: lowering of strength and stiffness of the material, or complete dissolution of the protruding components. This can be caused by the material degrading (lowering of molecular weight), softening of material (weakening of intermolecular bonds), swelling/expansion of material (binding of water) etc.

When the capsule device enters the sections of the more constricted parts of the gastric system (i.e. intestines or ileocecal valve) and tissue is potentially tight around the capsule device, the protruding components will be weak/soft enough to deflect potential force between device and tissue, avoiding any damage to the tissue.

Possible materials may be provided, but not limited to, PVA, co-polymers of PCL/PDS/PLA, rigid hydrogels or any dissolvable material that can be manufactured into a solid form (e.g. compressed powder). In alternative embodiments, algae-based materials may also be used.

In alternative embodiments, the components may be designed differently, so that only one of the components are required to swell, soften or degrade—mainly the outermost components—which can then shield remaining still rigid components. An example of this, could be to coat the outermost component in a hydrogel that swells several times its original geometry. Alternatively, the outermost components are made solely of a material that swells several times its original size, but doesn't degrade in a biological environment. The said methods for providing safe passage of the actuated capsule device may be used in combination with any of the embodiments described in the present disclosure.

Although the above description of exemplary embodiments mainly concerns ingestible capsules for delivery in the stomach, the present deployment principle generally finds utility in capsule devices for lumen insertion in general, wherein a capsule device is positioned into a body lumen for deployment of a delivery member, or other tissue interfacing components, such as sensors configured as monitoring devices. Non-limiting examples of capsule devices in accordance with aspects of the present invention may, apart from the stomach administered devices discussed above, include capsule devices for intestinal delivery of a drug by delivery into the tissue wall of an intestinal lumen, such as a lumen of the small intestines or a lumen of the large intestines.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An ingestible device suitable for swallowing into a lumen of a gastrointestinal tract of a patient, the lumen having a lumen wall, the ingestible device comprising:
a housing defining an internal hollow and comprising a stop geometry arranged within the internal hollow and an exit hole,
a tissue penetrating member disposable in the housing, the tissue penetrating member having a tissue penetrating first end, a second end opposite the first end and a radially outwards facing surface between the first end and the second end, and
an actuator arrangement comprising:
a) a pushing portion configured for movement along an axis from a first position and into a second position, the pushing portion being configured for providing a force onto the tissue penetrating member to move the tissue penetrating member from an initial position within the housing to a lodged position where at least a portion of the tissue penetration member is external to the housing and at least partially lodged in tissue of the lumen wall, and
b) a holder portion coupled with the pushing portion for initial axial movement with the pushing portion, wherein when the pushing portion assumes the first position the holder portion releasably holds the tissue penetrating member in axial retaining engagement,
wherein, as the pushing portion moves towards the second position, the tissue penetrating member is moved into tissue until the holder portion enters into engagement with the stop geometry of the housing thereby axially stopping the holder portion, whereafter the pushing portion moves further to release the axial retaining engagement and advance the tissue penetrating member into its lodged position.

2. The ingestible device as in claim 1, wherein, when the tissue penetrating member assumes the initial position, the holder portion assumes a start position, and wherein the holder portion is moved from the start position towards the stop geometry by slaved movement relative to the pushing portion.

3. The ingestible device as in claim 2, wherein the holder portion, when assuming the start position, releasably engages the pushing portion, such as by a friction engagement or a snap engagement, and wherein the pushing portion releases from engagement with the holder portion when the holder portion enters into engagement with the stop geometry.

4. The ingestible device as in claim 2, wherein the holder portion is formed as a sleeve that, when the holder portion is slaved relative to the pushing portion, interconnects the pushing portion with the tissue penetrating member.

5. The ingestible device as in claim 1, wherein the holder portion comprises at least one radially resilient gripping member providing a radially inwards directed force onto the tissue penetrating member, wherein the radially resilient gripping member cooperates with the stop geometry of the housing to release the radially inwards directed force upon the holder portion engaging the stop geometry.

6. The ingestible device as in claim 1, wherein the holder portion defines a distal facing end surface and wherein, when the holder portion holds the tissue penetrating member in axial retaining engagement, the distal facing end surface at least partially encircles the tissue penetration member while the tissue penetrating member extends distally from the distal facing end surface of the holder portion.

7. The ingestible device as in claim 1, wherein the holder portion comprises at least one piercing portion that protrudes axially from the distal facing end surface, the at least one piercing portion extending axially past the first end of the tissue penetrating member.

8. The ingestible device as in claim 1, wherein the housing comprises an exterior surface portion surrounding the exit hole, wherein the exit hole permits the tissue penetrating member, the holder portion and the pushing portion to protrude through the exit hole, and wherein the pushing portion in its second position pushes the first end of the tissue penetrating member a predefined distance from the exterior surface portion, said pre-defined distance selected between 3 and 7 mm.

9. The ingestible device as in claim 1, wherein, when the tissue penetrating member assumes the initial position, the tissue penetrating first end is axially separated relative to an exterior surface portion surrounding the exit hole by a separating distance, thereby enabling the tissue penetrating member to be advanced towards tissue at a target location by an acceleration stroke corresponding to the separating distance.

10. The ingestible device as in claim 1, wherein the pushing portion and the holder portion comprises a protruding section configured to protrude through the exit hole, and wherein at least a portion of the said protruding sections are made from a material configured to change shape, such as by degrading, softening or swelling, when exposed to a biologic fluid, such as gastric fluid.

11. The ingestible device as in claim 1, wherein the tissue penetrating member is a solid formed partly or entirely from a preparation comprising a therapeutic payload, wherein the tissue penetrating member is made from a dissolvable material that dissolves when inserted into tissue of the lumen wall to deliver at least a portion of the therapeutic payload into tissue.

12. The ingestible device as in claim 1, wherein an exterior portion of the tissue penetrating member defines an enclosure, and wherein a preparation comprising a therapeutic active substance forms a liquid, gel or powder accommodated within the enclosure.

13. The ingestible device as in claim 1, wherein the actuator arrangement comprises an energy source configured for powering the tissue penetrating member for being advanced from the housing and into the lodged position in the lumen wall and wherein a trigger arrangement is coupled to the actuator arrangement for initiating release of energy from the energy source thereby driving the pushing portion from the first position to the second position.

14. The ingestible device as in claim 13, wherein the energy source comprises a drive spring, such as a compression spring or a tension spring, the spring being strained or configured for being strained so as to provide an axial load on the pushing portion for driving movement of the pushing portion from the first position to the second position.

15. The ingestible device as in claim 1, wherein the ingestible device is configured as a self-orienting capsule device, wherein when the self-orienting capsule device is at least partially supported by the tissue of the lumen wall, the self-orienting capsule device orients in a direction to allow the tissue penetrating member to be inserted into the lumen wall.

16. The ingestible device as in claim 8, wherein the pre-defined distance selected is between 4 and 6 mm.

17. The ingestible device as in claim 8, wherein the pre-defined distance selected is between 4.5 and 5.5 mm.
